(19) United States
(12) Patent Application Publication
(10) Pub. No.: US 2018/0263536 A1
(43) Pub. Date: Sep. 20, 2018

(54) CHANGES IN AUDITORY EVOKED RESPONSES AS SIMPLE, RAPID BIOMARKERS FOR BLAST INJURY AND OTHER TRAUMATIC BRAIN INJURIES

(71) Applicant: Purdue Research Foundation, West Lafayette, IN (US)

(72) Inventors: Riyi Shi, West Lafayette, IN (US); Edward Lee Bartlett, West Lafayette, IN (US); Jesyin Lai, West Lafayette, IN (US); Nicholas Stephen Race, Indianapolis, IN (US)

(73) Assignee: Purdue Research Foundation, West Lafayette, IN (US)

(21) Appl. No.: 15/923,316

(22) Filed: Mar. 16, 2018

Related U.S. Application Data
(60) Provisional application No. 62/472,732, filed on Mar. 17, 2017.

(51) Int. Cl. A61B 5/12; A61B 5/00

(52) U.S. Cl. CPC A61B 5/125; A61B 5/4064

(57) ABSTRACT

Hearing difficulties are the most commonly reported disabilities among Veterans. Blast exposures during explosive events likely play a role, given their propensity to directly damage both peripheral auditory system (PAS) and central auditory system (CAS) components. Post-blast PAS pathophysiology has been well-documented in both clinical case reports and laboratory investigations. In contrast, blast-induced CAS dysfunction remains under-studied, but has been hypothesized to contribute to an array of common Veteran behavioral complaints including learning, memory, communication, and emotional regulation. This investigation compared the effects of acute blast and non-blast acoustic impulse trauma in adult male Sprague-Dawley rats. An array of audiometric tests were utilized, including distortion product otoacoustic emissions (DPOAE), auditory brainstem responses (ABR), middle latency responses (MLR), and envelope following responses (EFR). Generally, more severe and persistent post-injury central auditory processing (CAP) deficits were observed in blast-exposed animals throughout the auditory neuraxis, spanning from the cochlea to the cortex. DPOAE and ABR results captured cochlear and auditory nerve/brainstem deficits, respectively. EFRs demonstrated temporal processing impairments suggestive of functional damage to regions in the auditory brainstem and the inferior colliculus. MLRs captured thalamocortical transmission and cortical activation impairments. Taken together, the results suggest blast-induced CAS dysfunction may play a complementary pathophysiologic role to maladaptive neuroplasticity of PAS origin. Even mild blasts can produce lasting hearing impairments that can be assessed with non-invasive electrophysiology, allowing these measurements to serve as simple, effective diagnostics.

### FIELD OF INVENTION

This disclosure relates to an integrated standalone device and the use of it for diagnosis of early brain injury. Particularly, the disclosure detects auditory evoked response changes as biomarkers for blast injury and other traumatic brain injuries.

### BACKGROUND

Blast injury, concussions, and other traumatic brain injuries are often characterized by diffuse damage that makes diagnosis difficult and challenging to measure without expensive and bulky technologies that are not available outside of medical or research settings. Even in advanced clinical settings, current technology does not reliably detect mild head injuries, which are by far the most common. Some new techniques have been tested in recent years including IMPACT psychomotor testing, advanced neuroimaging, and integrated helmet sensors. Neuroimaging has not established consistent clinical criteria that substantiate the diagnosis of a mild brain injury. Further, neuroimaging requires large, room-sized scanners. Due to practical obstacles including cost, time, staff, training level, and portability, these sophisticated scanners are not scalable to a population of meaningful size nor portable for ‘field’ use.

Integrated helmet sensors have proven unreliable and, further, there are not validated criteria that distinguish ‘injured’ from ‘uninjured’. IMPACT testing also has flaws. Because it is based on behavioral performance, pre-injury baseline performance can be artificially reduced (intentionally faked) by subjects to make ‘medical clearance’ easier to achieve after injury. What is lacking are objective diagnostics indicative of multi-region brain injury that can be used in a variety of field conditions and for persons with limited training, such as in military field operations, sports sidelines, ambulances, and emergency rooms.

It is clear that current technologies are lacking a sensitive, objective, portable diagnostic tool that requires minimal training for use. This disclosure provides an alternative to address this need.

### SUMMARY OF THE INVENTION

This disclosure provides a standalone device for evaluating brain injury. The device comprises an apparatus to provide and measure a controlled sound stimuli, and at least one single or multichannel auditory evoked potential recordings of synchronized neural activities selected from the group consisting of distortion product otoacoustic emissions (DPOAE), auditory brainstem responses (ABRs), envelope following responses (EFRs), and middle-latency responses (MLRs).

In some embodiment the aforementioned standalone device provided controlled sound stimuli is selected from noises comprising simple clicks and pure tones, and temporally modulated sounds.

This disclosure further provides a method of evaluating brain injury in a patient. The method comprises the steps of:
a. using a standalone device to measure the baseline single or multichannel auditory evoked potential recording of synchronized neural activities selected from the group consisting of distortion product otoacoustic emissions (DPOAE), auditory brainstem responses (ABRs), envelope following responses (EFRs), and middle-latency responses (MLRs);
b. monitoring the patient until a sound event occurs;
c. using the standalone device to measure the post event auditory evoked potential recording of synchronized neural activities identified in step a for an extended time point;
d. determining the auditory evoked potential recording differences for DPOAE, ABRs, EFRs and MLRs between the baseline and post event time point; and
e. correlating DPOAE, ABRs, EFRs and MLRs data associated brain region to determine the extent of brain injury.

In some embodiment the aforementioned method may provide brain injury related to brain stem, thalamus and cortex.

In some embodiment the aforementioned method further comprising examining acrolein-adduct in auditory cortex at about day 2 and about day 7 post event, wherein increased acrolein in auditory cortex and auditory thalamus indicates brain injury.

This disclosure further provides a method of non-invasively evaluating a patient's post event auditory recovery. The method comprises:
providing a sound speech to the patient at various time point after the event;
obtaining a waveform or a spectrogram of the patient;
ascertaining a clear iteration and/or pitch salience of each sound speech provoked; and
recording the time point when a peak iteration and/or a pitch salience is identified.

In some embodiment the aforementioned time point is about 7 days post a blast.

These and other features, aspects and advantages of the present invention will become better understood with reference to the following figures, associated descriptions and claims.

### BRIEF DESCRIPTION OF THE DRAWINGS

FIG. 1: Statistics color map key. Color map to be used to illustrate p-values for data. Grayscale colors indicate non-significant effect (p>0.01). ‘Hot’ colors (yellow to red) indicate significant effect (p<0.01), with red indicating values<0.001. Boxes outlined in black are between-group comparisons at each time point (Blast vs. Noise). Red and blue boxes indicate within-group comparisons at different time points for Blast (red) and Noise (blue), respectively. Pre: pre-exposure to blast or noise; 2 wk: 2 weeks after exposure; 1 mo: 1 month after exposure.

FIG. 2: No impairments observed for lower-frequency DPOAEs. 2 kHz and 4 kHz DPOAEs indicated similar pre- and post-injury I/O function generation for both Blast and Noise animals. No significant effects of group (Blast vs. Noise) or time point (Pre vs. 2 wk vs. 1 mo) or Group*Time Point interaction were present for 2 kHz (top) or 4 kHz (bottom) pure tones. Data shown as mean±SEM.

FIG. 3: Blast animals demonstrated more severe and persistent deficits on higher-frequency DPOAEs. 8 kHz and 12 kHz DPOAEs revealed group and time-point dependent I/O function differences. (top) Pure tones set at 8 kHz. Both Blast and Noise animals demonstrated significant differences at 2 weeks post-injury from pre-injury recordings. At 1 month post-injury, only Blast group recordings were significantly lower (worse) than pre-injury, at which point between-group differences also emerged. (bottom) Pure tones set at 12 kHz. Only Blast group animals demonstrated significant reductions (worse) compared to pre-injury recordings at both the 2 week and 1 month post-injury time points. Between-group differences were also present at both 2 weeks and 1 month with Blast group results lower (worse) than the Noise group. Data shown as mean±SEM.

FIG. 4: Blast animals experienced sustained alterations in click ABR thresholds for at least 1 month, while Noise animals appear unaffected. Click ABR threshold changes indicate a significant group effect, with Blast group thresholds significantly higher (worse) than the Noise group. A time point effect was also present for the Blast group with both 2 week and 1 month post-exposure thresholds significantly higher (worse) than pre-injury recordings. Noise group animals did not exhibit any time-dependent significant alterations in click ABR thresholds. Blast thresholds were significantly higher (worse) than Noise thresholds at both post-injury time points. Data shown as mean±SEM.

FIG. 5: Tone ABR thresholds demonstrated trends of threshold increases (worse) for Blast and decreases (better) for Noise animals at high frequencies. No consistent statistically significant effects were observed for tone threshold measurements, except at 12 kHz. However, Blast group recordings show trends of threshold elevation (worse) for higher frequencies at both post-injury time points. Noise group threshold changes were minimal at 2 weeks post-injury, but 1 month recordings demonstrated a trend of decreased (better) thresholds for tones≥8 kHz. Data shown as mean±SEM.

FIG. 6: 80 dB pSPL click ABRs revealed reduced (worse) wave I, IV, and V amplitudes post-injury to a greater degree in Blast animals. Blast animals demonstrated significant wave I (top), IV (middle) and V (bottom) amplitude decreases (worse) at both 2 weeks and 1 month post-injury when compared to pre-injury recordings. Waves IV and V were significantly decreased in Noise animals, but only at the 1 month post-injury time point. Wave I recordings captured significant differences between Blast and Noise groups at 2 weeks post-injury, showing lower (worse) amplitudes in the Blast group. Wave IV and V amplitudes demonstrated non-significant trends of difference, again with Blast group having lower (worse) amplitudes. Data shown as mean±SEM.

FIG. 7: Post-injury 30 dB SL click ABRs revealed wave I, IV, and V amplitudes reductions (worse) in Blast but not Noise animals. Blast animals demonstrated significant decreases (worse) in wave I (top), IV (middle) and V (bottom) amplitudes at both 2 weeks and 1 month post-injury when compared to pre-injury recordings. No significant pre- vs. post-injury changes were observed for Noise animals. Wave I recordings captured the only significant difference between Blast and Noise groups at 2 weeks post-injury, although wave IV and V amplitudes demonstrated trends of difference. Data shown as mean±SEM.

FIG. 8: Deficits observed on toneburst ABRs were less apparent than on click ABRs. Significant differences were observed using 80 dB pSPL 8 kHz toneburt ABRs between Blast and Noise groups at 2 weeks post-injury with similar overall trends to click ABR results. Blast animals demonstrated trends of decrease (worse) in 2 week post-injury wave I (top), IV (middle) and V (bottom) amplitudes that appeared to flatten and sustain to 1 month post-injury, but changes were not significant compared to pre-injury results. Trends in wave amplitude decreases (worse) in Noise animals that occurred between 2 weeks and 1 month post-injury, were also non-significant. Wave I amplitudes captured the only significant difference between Blast and Noise groups at 2 weeks post-injury. Data shown as mean±SEM.

FIG. 9: Click and tone MLRs captured more apparent thalamic and cortical deficits in Blast than Noise animals. MLRs were elicited by clicks (A,B,E,F) or brief 8 kHz tones (C,D,G,H) at 80 dB pSPL. Minimal pre- vs. post-injury differences were observed in Noise animals (A-D). Pre- vs. post-injury differences were significant in Blast animals (E-H) for the early wave components corresponding to ABR generator regions (<10 msec) as well as two later MLR components (15-25 msec and 35-48 msec). Changes in the later MLR components observed in Blast animals corresponded to altered thalamic, thalamocortical, and cortical activation. Gray boxes indicate time windows in which significant differences were observed between the pre- and post-injury waveforms, based on comparison of 5 msec moving averages (1 msec steps) and using the Wilcoxon rank-sum test (p<0.05). Data shown as mean (solid lines) and mean+SEM (dashed lines).

FIG. 10: EFRs captured central subcortical processing impairments in the IC with possible cortical contribution. 8 kHz 80 dB SPL SAM EFRs showed significantly decreased (worse) EFR amplitudes at lower AMFs (≤64 Hz), corresponding to IC and possibly cortex, at 2 weeks and 1 month post-injury to a greater degree in Blast than Noise animals. Mid-range AMF EFRs (90-512 Hz) showed trends of decrease (worse) for Blast, but not Noise animals, while higher AMF EFRs (>512 Hz) showed minimal changes in both groups, suggesting more peripheral EFR remained intact. Data shown as mean±SEM.

FIG. 11: Summary schematic of blast-induced auditory pathophysiological findings and implications from the cochlea to the cortex. Auditory system drawing adapted with permission from Caspary et al. (2008). Abbreviations: ABR=auditory brainstem recording, MLR=middle latency recording, EFR=envelope following response, DPOAE=distortion product otoacoustic emission, AMF=amplitude modulation frequency, OHC=outer hair cell, IHC=inner hair cell, AN=auditory nerve, DCN=dorsal cochlear nucleus, SOC=superior olivary complex, LL=lateral lemniscus, IC=inferior colliculus, MGB=medial geniculate body, A1=primary auditory cortex.

FIG. 12: Oxidative stress after blast. Acrolein-lysine adducts elevated in auditory cortex and auditory thalamus (slice F) bilaterally.

FIG. 13 Top: Waveform and spectrogram of the speechlike sound used to probe complex sound representations. It is a rising pitch of the vowel /ee/, as in “Is this a bee?” or the rising tone 2 Chinese “yi.” Bottom: Example auditory evoked potential waveforms 7 days post-noise (blue) or blast (red) exposure for an increase in the iterations or pitch salience going from top to bottom. In all cases, the blue waveform is larger and much closer to the stimulus waveform than the blast exposed responses.

### DETAILED DESCRIPTION

While the concepts of the present disclosure are illustrated and described in detail in the figures and the description herein, results in the figures and their description are to be considered as exemplary and not restrictive in character; it being understood that only the illustrative embodiments are shown and described and that all changes and modifications that come within the spirit of the disclosure are desired to be protected.

Unless defined otherwise, the scientific and technology nomenclatures have the same meaning as commonly understood by a person in the ordinary skill in the art pertaining to this disclosure.

With increases in explosive device usage for modern conflict, blast injuries have unfortunately gained notoriety as warfare's hallmark injury (Rosenfeld et al. 2013). Advances in military protective equipment have increased the survival rate of individuals exposed to blast, permitting exposure to higher intensity shock waves before mortality thresholds are reached (Bass et al. 2008, Bass et al. 2012, Rafaels et al. 2011). Due to increased blast survivability, new organ systems have emerged as vulnerable to blast, particularly the brain and auditory system. Even prior to the advent of modern protective equipment, damage to the ear historically has been the most commonly reported consequence of blast exposure (Sharpnack et al. 1991). Among Veterans with service-connected disabilities, tinnitus and hearing loss are the two most prevalent conditions (Affairs USDoV, 2014). Given the recently discovered vulnerability of the brain to blast (Rafaels et al. 2011, Rosenfeld et al. 2013, Song et al. 2015, Walls et al. 2015) and known susceptibility of the peripheral auditory system (DePalma et al. 2005, Sharpnack et al. 1991), it is likely that repeated exposure to blast events incites significant damage at all levels of the auditory pathway and is a major factor in the high prevalence of hearing impairment in Veterans.

Traditionally, post-blast hearing impairment has mostly been attributed to peripheral damage to the eardrum, middle ear, and inner ear (Kerr 1980). Eardrum rupture was once used to identify blast-injured individuals, but was determined to be a poor diagnostic indicator due to a high rate of false negatives (DePalma et al. 2005, Ritenour et al. 2008). Recent clinical (Berger et al. 1997, Bressler et al. 2016, Cave et al. 2007, Cohen et al. 2002, Gallun et al. 2012, Lew et al. 2009, Ritenour et al. 2008, Saunders et al. 2015) and laboratory (Cho et al. 2013b, Du et al. 2013, Ewert et al. 2012, Luo et al. 2014 a,b, Mahmood et al. 2014, Mao et al. 2012, Patterson and Hamernik 1997) investigations have reported that both the peripheral (middle and inner ear) and central (brainstem and brain) auditory systems (PAS and CAS, respectively) are vulnerable to blast injuries, even at relatively mild intensities that could be encountered by a large proportion of personnel. Such knowledge warrants deeper understanding of the pathology of post-trauma auditory dysfunction, particularly deficits that stem from CAS damage, an area with little research effort to date. Furthermore, given the specialized signaling and energetic demands necessary to permit the exquisite temporal precision (Joris et al. 2004) and sound source localization (Brand et al. 2002, Grothe et al. 2010) of the auditory system, assessments of auditory processing may act as a sensitive biomarker more generally for central nervous system function. Finally, no effective therapies for auditory damage have been established, leading to a great deal of ongoing suffering for active military, Veterans, and civilians exposed to blast events.

In addition to observations of tympanic membrane (eardrum) rupture in combat (DePalma et al. 2005), clinical reports have consistently documented lasting hearing impairment and tinnitus in Veterans (Cohen et al. 2002, Cave et al. 2007, Ritenour et al. 2008, Saunders et al. 2015). Recent laboratory investigations have demonstrated the capacity of blast injury to damage both inner and outer hair cells (IHC and OHC) in the cochlea (Cho et al. 2013b, Ewert et al. 2012, Patterson et al. 1997), which could lead to permanently elevated thresholds. However, other studies have demonstrated that sound exposure leading to slight temporary shifts in thresholds can still lead to lasting auditory and perceptual deficits. In support of this notion, it was recently reported for humans exposed to blasts that behavioral deficits are common despite relatively small threshold shifts and little change in auditory brainstem responses (Bressler et al. 2016, Gallun et al. 2012, Saunders et al. 2015). In these cases, cochlear ultrastructure is relatively intact, but the number of synapses between hair cells and auditory nerve (AN) fibers is greatly reduced (Cho et al. 2013b). This suggests that many of the military personnel exposed to blast but deemed within the normal range by threshold testing may in fact have hearing impairment, stemming from compromised processing or perception, that could impact their performance in military operations and their return to civilian life (Bressler et al. 2016, Gallun et al. 2012, Lew et al. 2009, Saunders et al. 2015). Further, PAS assessments alone may inadequately capture the immediate and lasting effects of blast exposure on the auditory system and behaviors which depend upon its intact function.

To date, few controlled laboratory studies of post-blast CAS pathophysiology have been performed. Some clinical reports have suggested that, over the long term, the principal lasting injuries to the auditory system may reside centrally rather than peripherally (Bressler et al. 2016, Gallun et al. 2012, Saunders et al. 2015). In rats exposed to blast injury, there have been reports of neuronal spontaneous hyperactivity in brainstem auditory nuclei (Luo et al. 2014a,b). Additionally, diffusion tensor imaging post-blast in rats showed changes in the inferior colliculus (IC) and auditory thalamus, but not the auditory cortex (Mao et al. 2012), suggesting the subcortical CAS may be particularly vulnerable to blast injury. Despite these findings, the pathophysiological mechanisms of blast injury to the auditory system and respective contributions of damage to the PAS vs. CAS remain unclear. This knowledge gap precludes targeted development of innovative diagnostic techniques, therapies, and protective technologies, though some therapies have been attempted with mixed results (Du et al. 2013, Ewert et al. 2012, Mahmood et al. 2014). Moreover, if CAS deficits can serve as sensitive biomarkers for more general central nervous system (CNS) neuronal function, they have the major advantage of being easily accessible for diagnostics. It has been widely demonstrated by our group and others that even mild blast exposures can diffusely injure the brain (Budde et al. 2013, Cernak et al. 2001, Cho et al. 2013a, Garman et al. 2011, Readnower et al. 2010, Walls et al. 2015), a finding consistent with the few existing reports of post-blast CAS dysfunction (Gallun et al. 2012, Luo et al. 2014a,b). In particular, our recent work has revealed that rapid, heterogeneous intracranial deformation during mild blast can deform and ultimately damage the brain via subsequent changes in oxidative stress levels, neuroinflammation, and blood-brain barrier (BBB) permeability—possible initiating mechanisms of altered CAS function (Song et al. 2015, Walls et al. 2015).

We have conducted a preliminary investigation into PAS and CAS dysfunction by examining differences between the effects of two different forms of acute acoustic trauma (AAT): a single mild blast exposure (shock wave+noise) to a single impulse noise-only acoustic trauma. Multiple measures of auditory processing were performed, including distortion product otoacoustic emissions (DPOAE), auditory brainstem responses (ABRs), middle latency responses (MLRs), and envelope following responses (EFRs). Collectively, the results of these tests suggest that post-blast functional impairments are present in the CAS for the processing of both simple (clicks and pure tones) and temporally modulated sounds throughout the auditory neuraxis. We demonstrate with the data presented here that auditory evoked potential recordings possess strong prospects for future diagnostic utility in identifying blast-injured individuals that are otherwise asymptomatic, a common clinical presentation among the >50% of blast-induced traumatic brain injuries (bTBI) classified as mild in severity (Galarneau et al. 2008, Tanielian and Jaycox 2008). Further, evoked potentials may aid in understanding basic pathophysiological mechanisms of neuronal dysfunction after blast injuries.

We have demonstrated that using a brief battery of sound stimuli, distortion product otoacoustic emissions (DPOAEs), and single-channel recording of synchronized neural activities in the forms of auditory brainstem responses (ABRs), envelope following responses (EFRs), and middle-latency auditory evoked potentials (MLAEPs) provides complementary information about multiple levels of the auditory neuraxis, from cochlea to cortex. In turn, these changes then provide correlated information regarding damage to neighboring non-auditory structures in brainstem, thalamus and cortex, among other regions.

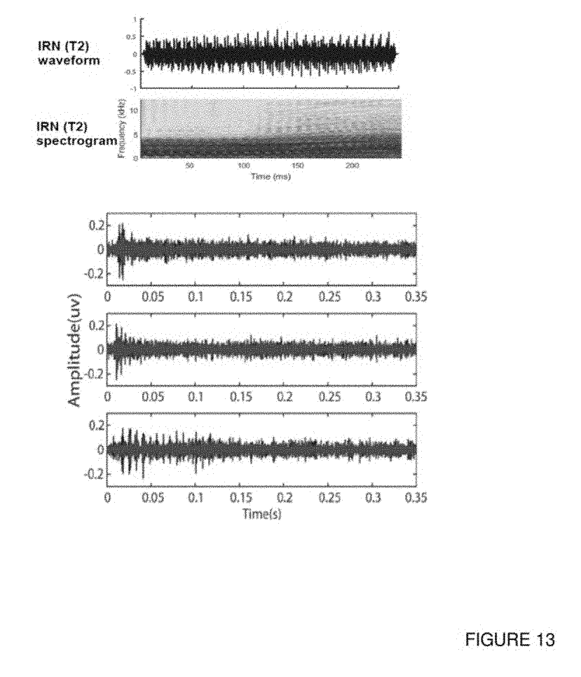

ABR threshold and amplitudes have shown that post-blast days 7-10 seem to be the period of rapid functional recovery from initial damage. Post-blast EFRs seem to show similar trends as we have observed in aged animals (FFR ratio increase but not significantly), but representations of speech like temporal fine structure seems to be significantly diminished, as shown using pitch contours generated by iterated rippled noise (IRN) waveforms, see FIG. 13, where the top panel shows waveform and spectrogram of the speech-like sound are used to probe complex sound representations. In the bottom panel of FIG. 13, example auditory evoked potential waveforms 7 days post-noise (blue) or blast (red) exposure are shown for an increase in the iterations or pitch salience going from top to bottom. In all cases, the blue waveform is larger and much closer to the stimulus waveform than the blast exposed responses.

In addition, we also have detected a significant elevation of acrolein-adduct in auditory cortex examined 2 and 7 days after blast trauma. Acrolein is a known neurotoxin capable of causing neurodegeneration.

Thus, we have provided further evidence that post-blast central hearing impairments detected by auditory evoked potentials are associated with brain tissue damage in auditory cortex. This further confirms the significance of detection of functional brain damage using noninvasive electrophysiological methods such auditory evoked potentials.

Together, this diagnostic battery has advantages over many existing diagnostic protocols. First, the equipment is simple and portable, similar to what is used nationwide in newborn hearing screening such that it does not require a large amount of specialized training for deployment. Second, though there is some overlapping information, the auditory brainstem responses, envelope following responses, and middle latency auditory evoked potentials give largely complementary information. Our proposed battery would include responses that measure both the brief and sustained neural responses, capturing onset and transmission responses, as well as more complex activity necessary for hearing and understanding human speech. Our work has demonstrated that auditory-evoked responses provide multi-faceted, multi-region information that can track the time course of injury and recovery and are correlated with damage in multiple brain regions.

Proposed herein is an integrated standalone device incorporating these unique stimulus profiles and processing algorithms as a simple, portable, ‘just press go’ rapid diagnostic tool easily deployable in a variety of settings. A point of distinction between this device and existing technology is that such a device will seamlessly integrate and administer a unique, configurable array of auditory stimuli that provide region-specific details regarding brain injury in an objective, quantifiable, and longitudinally-monitored fashion. This will not only answer the question of if a person has a brain injury, but also where in the brain and to what extent. This provides several advantages over current methods. We find this particularly applicable to the military setting, where blast injury is known to damage both the ears and brain. Using such a device and tests associated with it for blast injury diagnosis and recovery monitoring represents a novel use application. Such tests could 1) set objective quantifiable baseline neurofunctional performance metrics for each individual, 2) identify brain-injured individuals after combat events, 3) distinguish blast from non-blast brain injuries, 4) identify mild injuries, which currently cannot be reliably determined, and 5) monitor recovery objectively without patient interference or intentional skewing. None of these 5 tasks are currently achievable. Perhaps the key advantage of this device and its stimuli/algorithms are that they allow for rapid assessment using neurological functional metrics, as opposed to measurements of applied force to the head, anatomical indicators, or other non-validated tools, which have not proven reliable. In fact, the DoD recently canceled a major project which had been testing integrated helmet pressure sensors for blast injury, as it failed to meet the primary endpoint: screen for/diagnose brain injuries from blast exposure.

### Subject

Sprague-Dawley male rats (3-4 months) were used in this study. The animals were assigned into two groups randomly: Noise (N=8) and Blast (N=10). Noise animals were exposed only to blast noise, while Blast animals were exposed to both blast noise and shock wave injury (described below). All the animals were kept and raised in relatively quiet and standard laboratory animal housing conditions. All protocols were approved by the Purdue Animals Care and Use Committee (PACUC #1111000280).

### Blast Exposure

Animals were anesthetized by ketamine/xylazine cocktail injected intraperitoneally (80 mg/kg and 10 mg/kg, respectively). Absence of eye-blink and paw-withdrawal reflexes were ensured prior to proceeding. After verifying depth of anesthesia, animals were placed on a platform beneath an open-ended shock tube to be exposed to the blast event, as described in our prior publications (Song et al. 2015, Walls et al. 2015). Briefly, a custom plexiglass housing was used for body protection to simulate protective effects of military body armor (Bass et al. 2009). A stereotaxic head frame with bite bar and ear bars (Kopf Instruments) was utilized to fix the head in place and prevent blast wind-induced head acceleration. For the Blast group, each rat's head was positioned beneath the open end of the shock tube such that the dorsum of the skull was the incident surface exposed to a composite blast (shock wave+blast wind). The exposure conditions were consistent with our prior publications, having a recorded pressure profile with a near-instantaneous rise to peak pressure, followed by overpressure and underpressure periods as follows: side on (static) 150 kPa maximum overpressure, 1.25 msec overpressure duration, and 20 kPa minimum underpressure; face on (dynamic) 160 kPa maximum overpressure, 1.75 msec overpressure duration, and 5 kPa minimum underpressure. These exposure conditions have been validated to correspond to a mild severity in this model, lacking acute motor and memory deficits or evidence of intracranial hemorrhage, while presenting evidence of subclinical elevations in oxidative stress, blood-brain-barrier permeability, and neuroinflammatory activity (Walls et al. 2015). Millimeter-scale point deformations in the brain have also been observed in this model (Song et al. 2015). For the Noise group, the animals were placed in a location equidistant from the source of the blast, but out of the path of the shock wave so as to only be exposed to loud sound from the blast event. Tympanic membrane integrity was verified for all animals after injury using a surgical microscope. One Blast animal was excluded due to bilateral tympanic membrane perforation.

### Auditory Evoked Potential Recordings

Auditory functions and thresholds of all animals were assessed neurophysiologically at time points of pre-exposure (baseline), 2 weeks post-exposure, and 1 month post-exposure. The measurements that were used in this study include DPOAEs, ABRs, MLRs, and EFRs.

### DPOAEs

DPOAEs are commonly used in clinics and in research to test the biomechanical gain functions of OHC in the cochlea (Shaffer et al. 2003). DPOAEs are elicited by the presentation of two simultaneous tones at a ratio of approximately 1.2 to the cochlea. Electromotility driven by prestin (a protein located on OHC wall; Ashmore et al. 2010, Dallos et al. 2008, Liberman et al. 2004) and mechanoelectrical transduction of stereocilia above the hair cells (Avan et al. 2013, Kennedy et al. 2005) are believed to be the two main active processes that produce DPOAE responses. All DPOAEs were performed in a 9′×9′, double walled acoustic chamber (Industrial Acoustics Corporation) using standard techniques similar to those reported in Lai and Bartlett (2015). To anesthetize animals, animals were treated with 4% isoflurane via inhalation in an induction chamber. They were then transferred to the manifold and maintained with 1.8-2% isoflurane on a water-circulated warming blanket (Kent Scientific) to keep animals' body temperature at 37° C. throughout the duration of the recording session. Stimulus presentation and recordings were performed using BioSig (Tucker Davis Technologies, TDT) in the acoustic chamber. An earpiece (Etymotic-10B), which contained a miniature low-noise microphone and two sound delivery tubes, were inserted into the right ear canal of animals. The two sound delivery tubes connected the earpiece to the sound sources, which are two multifunction closed field speakers (TDT) that delivered pure tones to the ear canal. The two speakers presented sounds while the microphone recorded DPOAEs from the ear canal simultaneously. The output of the microphone was delivered as an input to a TDT RZ5 system that converted the recorded responses from analog to digital. Each response is an average response of 100 stimulus sweeps presented continuously to the animals. DPOAE input/output functions using two pure tones (f1 and f2, f2/f1=1.2) centering at 2, 4, 8 or 12 kHz were tested in all animals and in all recording sessions. For 2 and 4 kHz, the intensity of f1 was varied from 40 to 85 dB SPL in 5-dB steps. For 8 and 12 kHz, the intensity of f1 was varied from 35 to 75 dB SPL in 5-dB steps. The intensity of f2 was set at 10 dB SPL below that of f1. Data were processed with high-pass (HP) and low-pass (LP) filters using cutoff frequencies (f_{c}) of 80 Hz and 13200 Hz, respectively, prior to analysis.

### ABRs

Hearing thresholds of 2, 4, 8, 12, 16, and 32 kHz were estimated in all animals via ABRs. ABRs were performed after DPOAEs. While animals were under 1.8-2% isoflurane, needle electrodes (Ambu) were inserted subdermally. The channel 1 positive electrode was placed along the midline of the head (mid-sagittal) oriented Fz to Cz. The channel 2 positive electrode was positioned C3 to C4 along the interaural line. The negative/inverting electrode (used with positive electrodes for both channels 1 and 2) was placed under the mastoid of the right ear ipsilateral to the inserted earpiece and speaker. A ground electrode was placed in the nape of the neck. These configurations are consistent with prior publications from our laboratory (Lai and Bartlett 2015; Parthasarathy and Bartlett 2011, 2012; Parthasarathy et al. 2014). After electrode placement, electrode impedances were confirmed to be less than 1 kΩ using a low-impedance amplifier (RA4LI, TDT). Animals were subsequently sedated by intramuscular injection of 0.2 mg/kg dexmedetomidine (Domitor). To avoid anesthetic effects on neural responses, isoflurane was taken off after injection. As anesthetic effects take about 10 min to wear off (data not shown), ABRs were performed 15 min after cessation of isoflurane. Dexmedetomidine is an alpha-adrenergic agonist which acts as a sedative and an analgesic (Ter-Mikaelian et al. 2007). It decreases motivation but preserves behavioral and neural responses in rodents (Ruotsalainen et al. 1997). After injection, animals still respond to pain and acoustic stimuli but are immobile for approximately 3 hrs of recording period.

Using directly in front of the animals' face as the reference for 0° azimuth, the stimulus was presented free-field to the right ear (90° azimuth) of animals from a calibrated speaker (Bowers and Wilkins) from a distance of 115 cm. The speaker was directly facing the right ear. Rectangular clicks (0.1 msec duration) and tone-pips (2 msec duration, 0.5 msec cos² rise-fall time) varying in sound levels from 95 to 5 dB peak SPL (pSPL) in 5-dB steps were used in ABR recording. The frequencies of tone-pips were varied from 1 to 32 kHz in one-octave steps and 12 kHz was included to improve resolution near the animal's most sensitive portion of the audiogram (Parthasarathy et al. 2014). All stimuli were presented in alternating polarity at 26.6 per second. A 20 msec acquisition window (0-20 msec) was used and each ABR was an average of a total of 1500 repetitions for each sound level (750 at each polarity). Data were processed with HP (f_{c}=30 Hz) and LP (f_{c}=3000 Hz) filters prior to analysis. The ABR threshold was defined as the minimum sound level that produced a distinct ABR waveform. The ABR amplitudes of waves I, IV and V from channel 2 were estimated as the amplitude of the wave from the baseline (a cursor was placed at the average of the noise floor on the waveform prior to the cochlear microphonic and another cursor was placed at the peak to estimate the wave amplitude) in BioSig.

### MLRs

MLRs were recorded using short click and tone stimuli presented at a slower rate (4/sec vs. 26.6/sec in ABRs) and with a recording window of longer duration (100 msec vs. 20 msec in ABRs). By increasing the inter-stimulus interval and recording window duration to 100 msec, this provides enough time for the stimulus-evoked neural responses to occur in the auditory midbrain, thalamus and cortex (Barth and Di 1991, Di and Barth 1992, Phillips et al. 2011, Suta et al. 2011, McGee and Kraus, 1996), while still capturing responses from ABR brainstem generators in parallel. As such, early components (<10 msec) of the waveform collected under the MLR acquisition settings correspond to responses from ABR generator regions, while later responses correspond to the aforementioned more central generators in the thalamus and cortex. Only MLRs recorded from the interaural line (channel 2) were analyzed. Rectangular clicks (0.1 msec) and brief 8 kHz tones (2 msec) of alternating polarity were used in MLR recording. Stimuli were presented at 4/sec and 1500 repetitions were collected over an acquisition time window of 100 msec to obtain an average response. The sound levels of clicks were set at 80 and 70 dB pSPL while the intensity of brief 8 kHz tones were at 80 dB pSPL. Data were processed with HP (f_{c}=300 Hz) and LP (f_{c}=3000 Hz) filters prior to analysis.

### EFRs

EFRs were recorded during the same recording session as ABRs and MLRs using the same electrodes and similar techniques to Parthasarathy et al. (2014). Two channels were used to record EFRs in this study because they were sensitive to a complementary range of amplitude modulation frequencies (AMFs) (Parthasarathy and Bartlett, 2012). Channel 1 (vertical configuration) is more sensitive to higher AMFs (90-2048 Hz) while channel 2 (horizontal configuration) is more sensitive to lower AMFs (8-90 Hz). Simultaneous recording EFRs from these two channels aids in analyzing a wider range of AMFs as well as identifying temporal processing deficits at various auditory subcortical regions. Stimuli used for EFRs were sinusoidally amplitude-modulated (SAM) 8 kHz tone carriers at various AMFs and 100% modulation depth with stimulus duration of 200 msec. The carrier frequency was set at 8 kHz because frequencies of 6-16 kHz span the most sensitive hearing region of rats (Parthasarathy et al. 2014) and 8 kHz is approximately at the center (in octave) of this region. Frequency 8 kHz is near the most sensitive region of normal rat audiogram (Parthasarathy et al. 2014). The acquisition window was 300 msec long, and each response was an average of 200 repetitions. The modulation frequency of the stimulus was varied from 16 to 2048 Hz in 0.5-octave steps. SAM 8 kHz stimuli were presented at 80 dB SPL. Prior to EFR amplitude analysis, data were passed through LP and HP filters. The HP filters were applied differentially to AMFs 16-24 Hz (f_{c}=12 Hz), 32-64 Hz (f_{c}=30 Hz), and ≥90 Hz (f_{c}=80 Hz). The LP filter was applied uniformly across data for all AMFs (f_{c}=3000 Hz). The amplitudes measured at the stimulus modulation frequencies after a fast-Fourier transform (FFT) of time-domain response waveforms were used as a measure of phase-locking for multi-group comparison. Swaminathan and Heinz (2012) demonstrated that speech sound recognition depends on envelope cues<64 Hz in quiet and in noise, as well as temporal fine structure cues (64-300 Hz). Only envelope responses were assessed for the 90-2048 Hz AMFs used in the present study, which cover the entirety of the reported behaviorally-relevant range (Swaminathan and Heinz, 2012).

### Statistics

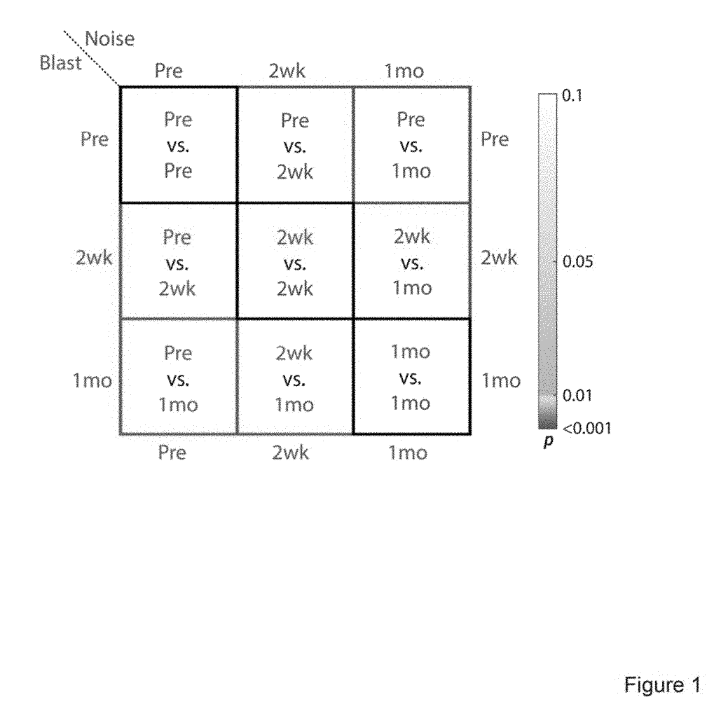

All statistics for DPOAE, ABR, and EFR measurements utilized a 3-way or 2-way repeated measures ANOVA test (α=0.01) to check the significance of each main effect and interaction. To improve efficiency of presentation and facilitate ease of data interpretation, a two-panel style will be utilized to illustrate the majority of the post-hoc Tukey' s LSM results, specifically for the primary outcome metric of interest: comparison of means for the Group*Time Point interaction. The left panel will illustrate the data sets via standard line plots or bar graphs, while the right panel will incorporate color maps to describe statistical results. A sample right panel with accompanying key is illustrated in FIG. 1.

Between-group comparisons (Blast vs. Noise) at a given time point (pre-exposure, 2 weeks, or 1 month) are shown in the black-outlined boxes along the diagonal. Within-group comparisons are designated by the colored-border boxes with red corresponding to Blast and blue corresponding to Noise groups. The comparisons in each box are documented explicitly in FIG. 1. The color of each box corresponds to the p-value for the comparison of interest, with grayscale values representing non-significant results (p>0.01) and ‘hot’ values (yellow to red) representing statistically significant results (p<0.01). Red indicates p<0.001 (close to 0). Directly in the text, p-value, F-statistic, degrees of freedom (reported as df1, df2 subscript on F-statistic), and effect size (ηₚ²) are reported.

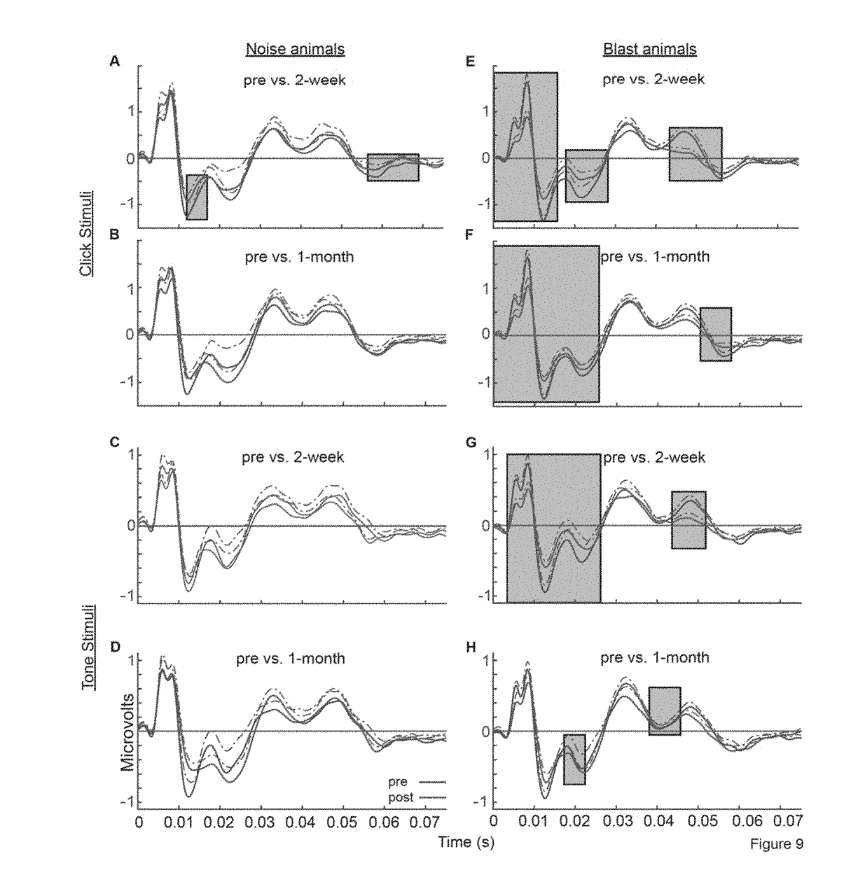

For MLR statistics, a 5 msec moving window (1 msec steps) of absolute amplitude was used to assess differences between pre-injury (blue) and post-injury (red) conditions at 2 weeks and 1 month post-exposure using the Wilcoxon rank-sum test. Only when two or more consecutive windows had p-values less than 0.05 was the window considered significant. Gray boxes in FIG. 9 indicate time windows in which significant differences were observed between pre- and post-injury waveforms.

### Results

### DPOAEs

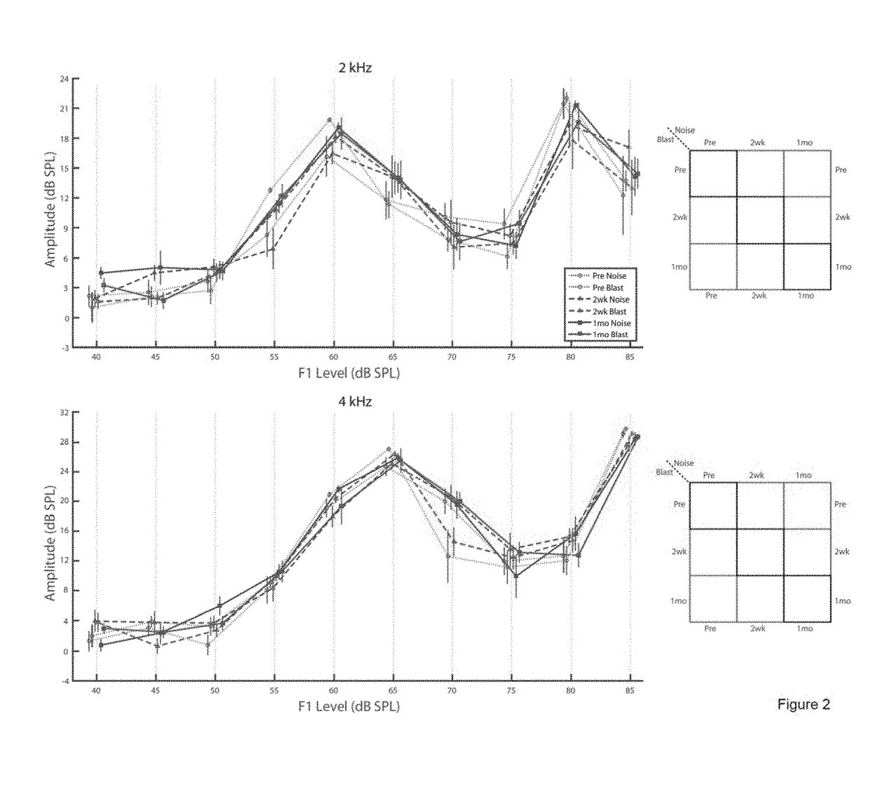

DPOAE recordings, which assess the integrity of OHC function (Shaffer et al. 2003), demonstrated frequency-dependent changes in input/output functions (I/O) after blast or noise exposure. Neither Blast nor Noise animals demonstrated significantly different I/O function amplitudes at 2 weeks or 1 month after injury for pure tones at 2 kHz or 4 kHz (FIG. 2). Main effects of Group (2 kHz: F_{1,12}=0.43|p=0.525|ηₚ²=0.03; 4 kHz: F_{1,12}=0.27|p=0.611|ηₚ²=0.02) and Time Point (2 kHz: F_{2,24}=2.09|p=0.146|ηₚ²=0.15; 4 kHz: F_{2,24}=1.98|p=0.160|ηₚ²=0.14) were not significant. Unsurprisingly, a significant main effect of Sound Level was observed (2 kHz: F_{9,108}=104.22|p<0.0001|ηₚ²=0.90; 4 kHz: F_{9,108}=267.73|p<0.0001|ηₚ²=0.96). No significant interaction effects were present.

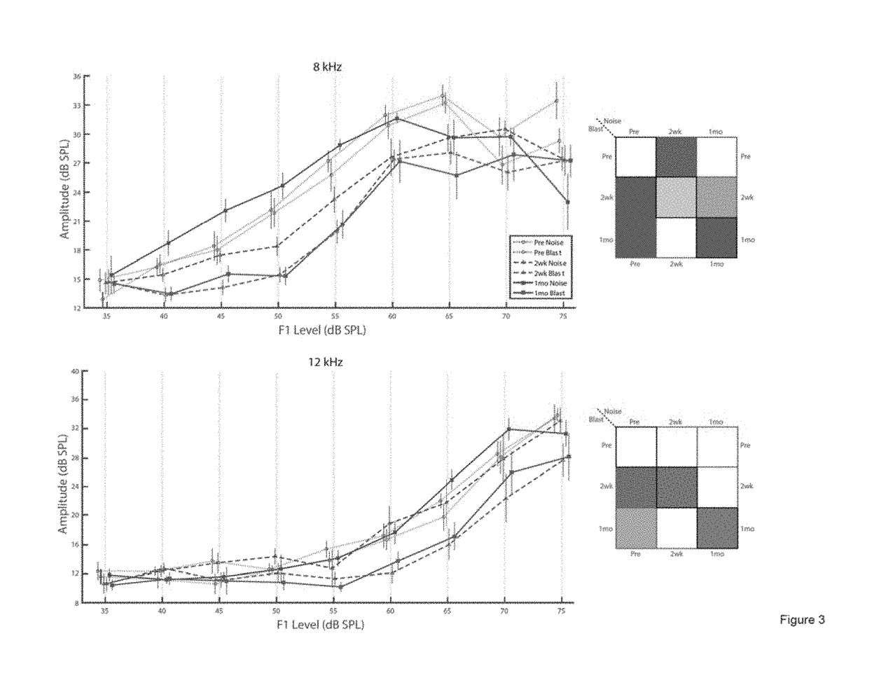

Conversely, both Blast and Noise animal I/O functions were altered post-injury for 8 kHz pure tones (FIG. 3, top). Significant main effects of Group (F_{1,12}=12.33|p=0.004|ηₚ²=0.51) and Time Point (F_{2,24}=20.10|p<0.0001|ηₚ²=0.63) were observed. Unsurprisingly, a significant main effect of Sound Level was also observed (F_{8,96}=120.54|p<0.0001|ηnₚ²=0.91). A significant Time Point*Sound Level interaction effect was also present (F_{16,192}=2.72|p<0.0006|ηₚ²=0.18). Blast animal I/O functions were significantly reduced (worse) to a similar degree compared to pre-exposure recordings at both 2 weeks and 1 month post-injury. Noise animals, however, only demonstrated significant reductions (worse) at 2 weeks post-injury. At 1 month post-injury, significant I/O function amplitude differences were observed between groups. Blast group amplitudes were significantly lower (worse) than Noise group amplitudes, which returned to statistical equivalence with Noise pre-injury recordings by the 1 month time point.

For 12 kHz pure tones (FIG. 3, bottom), only Blast group DPOAE I/O amplitudes were significantly different from pre-exposure recordings at 2 weeks and 1 month post-injury, showing reduced (worse) amplitudes at both time points. Noise group recordings did not demonstrate any time-dependent changes. Significant main effects of Group (F_{1,12}=12.08|p=0.005|ηₚ²=0.50), but not Time Point (F_{2,24}=3.55|p=0.045|ηₚ²=0.23) were observed. Consistent with this observation, significant differences between groups were observed at both 2 weeks and 1 month post-injury. Unsurprisingly, a significant main effect of Sound Level was observed (F_{8,96}=124.00|p<0.0001|ηₚ²=0.91). No significant interaction effects were present. Overall, our DPOAE results are consistent with the previous report that more apical, lower-frequency OHCs (2 and 4 kHz) are damaged by blast exposure to a lesser degree than more basal, higher-frequency OHCs (8 and 12 kHz) (Cho et al. 2013b).

### ABR Thresholds

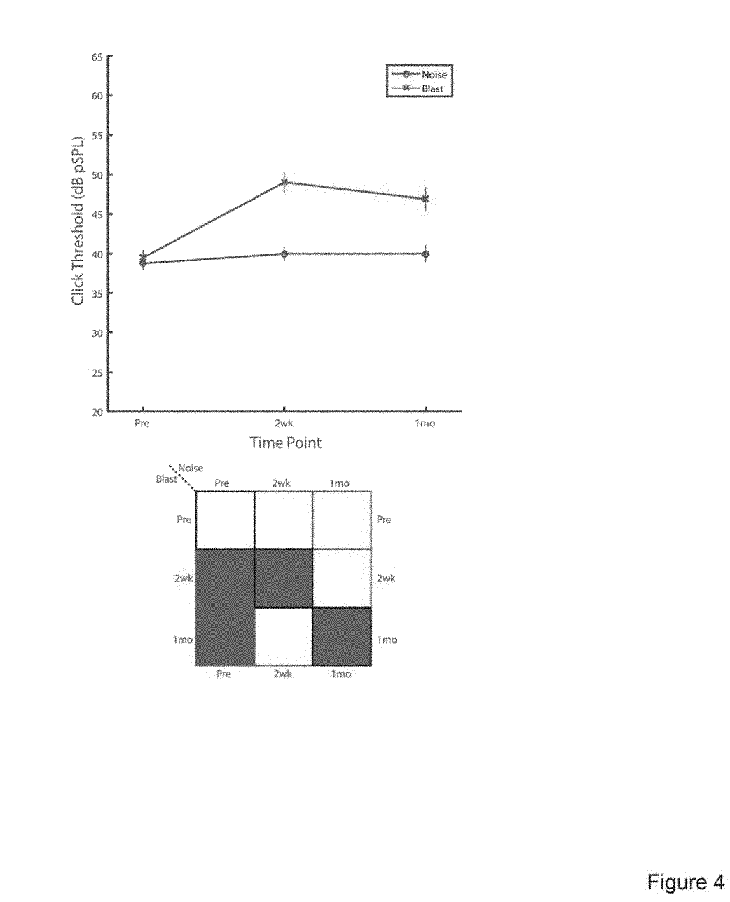

Click ABR recordings captured significant threshold differences for Blast, but not Noise, animals at both post-injury time points when compared to pre-exposure recordings (FIG. 4). Preliminary testing showed that both Blast and Noise groups had significant threshold shifts of >50 dB at 48 hours post-exposure (not shown), making it very difficult to evoke any measurable response in most animals. Thresholds for Blast group animals remained significantly elevated (worse) at both 2 weeks and 1 month post-injury, while Noise group animals did not demonstrate any observable changes in click thresholds (FIG. 4). Significant main effects of Group (F_{1,16}=30.50|p<0.0001|ηₚ²=0.66) and Time Point (F_{2,28}=13.16|p<0.0001|ηₚ²=0.48) were observed, in addition to a significant Group*Time Point interaction effect (F_{2,28}=7.55|p=0.002|ηₚ²=0.35).

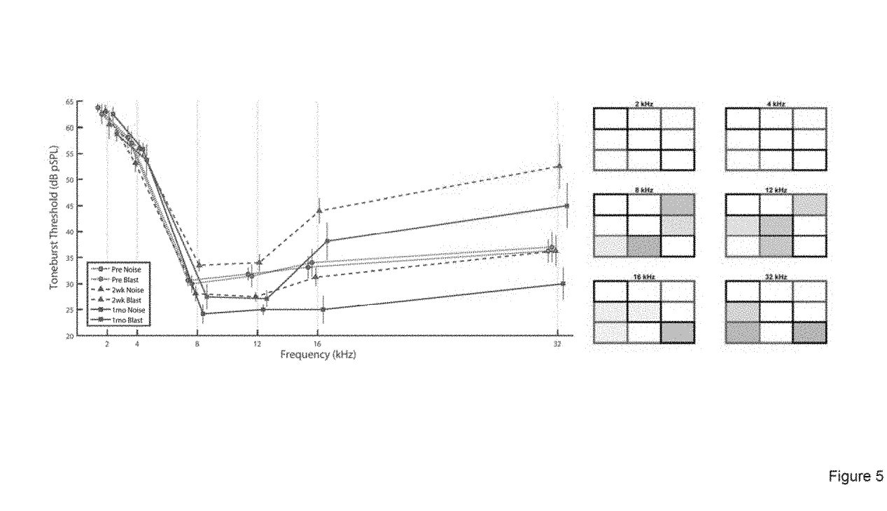

Tone ABR threshold shifts did not demonstrate consistent statistically significant effects for either Blast or Noise group animals at any of the frequencies tested between 2-32 kHz at any post-injury time point (FIG. 5). Unsurprisingly, a significant main effect of Frequency was observed (F_{5,80}=69.26|p<0.0001|ηₚ²=0.81), but main effects of Group (F_{1,16}=1.66|p=0.215|ηₚ²=0.09), Time Point (F_{2,28}=2.55|p=0.096|ηₚ²=0.15), and all interaction effects were non-significant. However, some informative trends were observed. High-frequency recordings (≥8 kHz) suggest potentially differential post-injury trends between groups, with Blast animals having slightly elevated (worse) thresholds (5-10 dB) while Noise animals had lower (better) thresholds when compared to pre-exposure recordings. In Blast animals, only 12 kHz hearing thresholds were significantly different between 2 weeks and 1 month post-injury, where hearing thresholds recovered at post-1-month and became lower (better), but not significantly, than pre-exposure. In addition, there were 10-15 dB increases (worse) in mean thresholds for frequencies≥16 kHz 2 weeks post-blast. Higher-frequency thresholds returned closer to pre-injury levels by 1 month post-blast (5-10 dB threshold increases: worse than pre-injury, but better than 2 weeks post-injury).

### ABR Wave Amplitudes

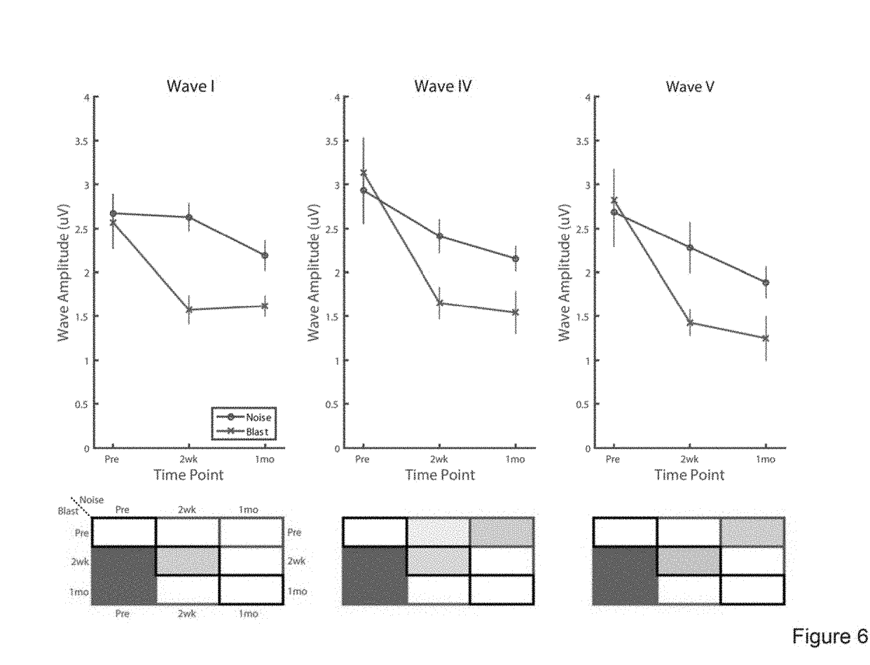

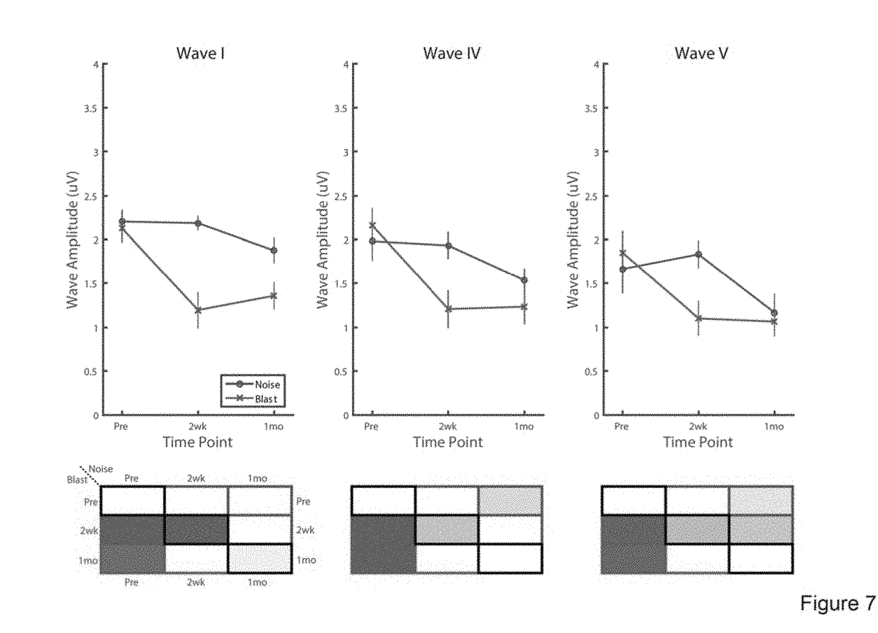

ABR wave amplitudes were assessed for waves I, IV, and V in response to click stimuli at 80 dB pSPL (FIGS. 6) and 30 dB sensation level (SL) (FIG. 7). For the 80 dB pSPL measurements (FIG. 6), significant main effects of Time Point (F_{2,24}=48.48|p<0.0001|ηₚ²=0.80) but not Group (F_{1,12}=2.74|p<0.124|ηₚ²=0.18) or Wave (F_{2,24}=2.52|p=0.101|ηₚ²=0.17) were observed. A significant Group*Time Point interaction effect was observed, however (F_{2,24}=10.25|p<0.0006|ηₚ²=0.46). For time points 2 weeks and 1 month post-injury, recordings were significantly lower (worse) than pre-exposure recordings for Blast animals for all waves. For Noise animals, significant reductions (worse) were present for waves IV and V only at 1 month post-injury when compared to pre-exposure recordings. These imply that injury-induced reduction (worse) of wave amplitudes happened at different rates in Blast and Noise animals. The only significant difference between groups was observed at 2 weeks post-injury for wave I.

In order to compensate for changes in threshold induced by blast, measurements were also made at 30 dB above each animal's individual threshold for clicks. For the 30 dB SL measurements (FIG. 7), significant main effects of Time Point (F_{2,28}=22.66|p<0.0001|ηₚ²=0.62) and Wave (F_{2,32}=8.33|p=0.001|ηₚ²=0.34) but not Group (F_{1,16}=3.94|p=0.065|ηₚ²=0.20) were observed. Additionally, a significant Group*Time Point interaction effect was observed (F_{2,28}=9.36|p<0.0008|ηₚ²=0.40). For time points 2 weeks and 1 month post-injury, amplitudes were significantly lower (worse) than pre-exposure in Blast animals for all waves. For Noise animals, no significant differences were observed between pre- and post-injury recordings. Consistent with the 80 dB pSPL measurements, the only significant difference between groups was observed at the 2 week post-injury time point for wave I, but trends of between-group differences were present for all waves at the 2 week time point.

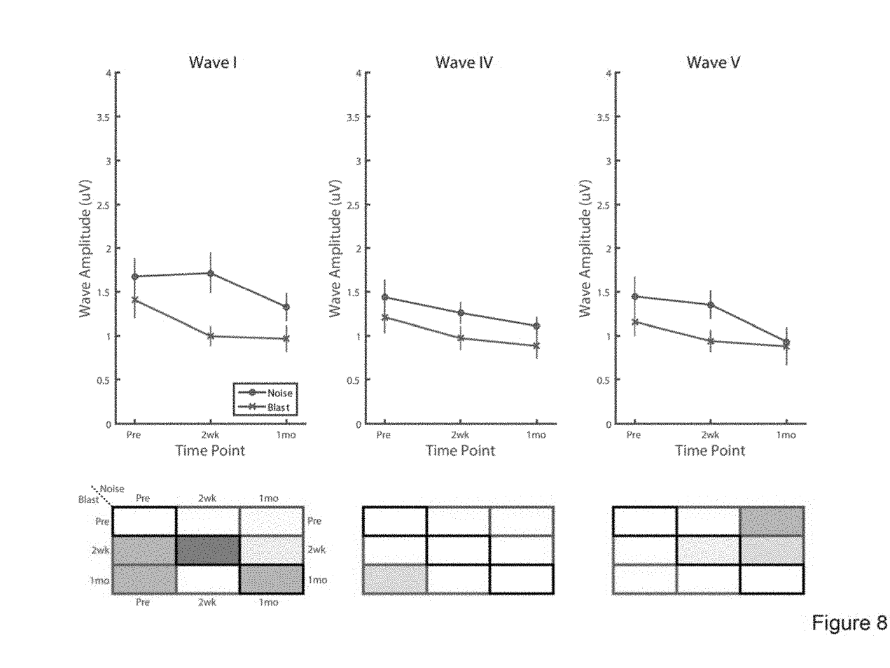

Tone ABR wave I, IV, and V amplitudes were also assessed in response to brief 8 kHz pure tones at 80 dB pSPL (FIG. 8). Significant main effects were observed for Time Point (F_{2,25}=12.00|p=0.0002|ηₚ²=0.49) and Wave (F_{2,32}=6.54|p=0.004|ηₚ²=0.29), but not Group (F_{1,16}=3.86|p=0.067|ηₚ²=0.19). No significant interaction effects were observed. Trends of decrease (worse) were observed for Blast animals at 2 weeks that sustained to 1 month post-injury and for Noise animals at 1 month, but none of the pre- vs. post-injury comparisons were significant for either group. Wave I amplitudes revealed a significant difference between Blast and Noise groups at the 2 week time point, where wave I amplitudes were significantly lower for Blast than for Noise animals. Similar to the threshold measurements, pure tones, at least at 8 kHz, seem to produce less consistent or dramatic reductions in ABR amplitudes post-injury compared to broadband clicks. On all click and tone ABRs, wave latencies were unchanged in all groups, at all time points, and for all ABR waves (not shown).

### Middle Latency Responses

Middle latency responses (MLRs) were measured using click or 8 kHz clicks and tones at 80 dB pSPL, similar to the ABRs. They were presented much more slowly (4 /sec MLR vs. 26.6/sec ABR) and measured over longer time windows (ranging from 10-50 msec) in order to track the impulse response to more central structures including the thalamocortical pathway. MLR grand average responses of Noise animals (FIG. 9A-D) versus Blast animals (FIG. 9E-H). A 5 msec moving window (1 msec steps) of absolute amplitude was used to assess differences between pre-injury (blue) and post-injury (red) conditions at 2 weeks and 1 month post-exposure. Gray boxes in FIG. 9 indicate time windows in which significant differences were observed between pre- and post-injury waveforms. Perhaps owing to the slower presentation rate, pre- and post-exposure differences were minimal for noise-exposed animals. For the Noise group (FIG. 9A-D), slight differences were apparent with click stimuli at 2 weeks post-injury, mainly in the 10-15 msec time window (FIG. 9A) corresponding to IC and thalamic excitation in rodents and humans (Barth and Di 1991, Kraus and McGee 1992, Kraus et al. 1992, McGee et al. 1991, Phillips et al. 2011). These differences were no longer significant by 1 month post-injury. Slight differences were noted in the 10-20 msec time window for other comparisons but did not reach statistical significance with the number of animals for comparison (N=6-8 for Noise animals). In contrast, there were clear significant differences in the ABR components of the waveform (<10 msec) under this slower stimulus presentation rate (4/s) in Blast animals (FIG. 9E-G), similar to results shown in FIGS. 6-8 at a faster presentation rate (26.6/s). Longer latency peaks corresponding to thalamocortical transmission and cortical activation (Barth and Di 1991, Brett et al. 1996, Kraus and McGee 1992, Kraus et al. 1992, McGee et al. 1991) also demonstrated significant differences in the Blast group (FIG. 9E-H). There were significant differences in the 15-25 msec window for both time points and both stimuli for Blast animals, corresponding to thalamocortical transmission (Di and Barth 1992). Finally, there were two late peaks in the MLR, at approximately 35 and 48 msec latencies. The second peak, corresponding to auditory cortical activation (Barth and Di 1991, Di and Barth 1992), was affected in Blast animals for both stimuli and time points, but this was not the case for Noise animals.

### Envelope Following Responses

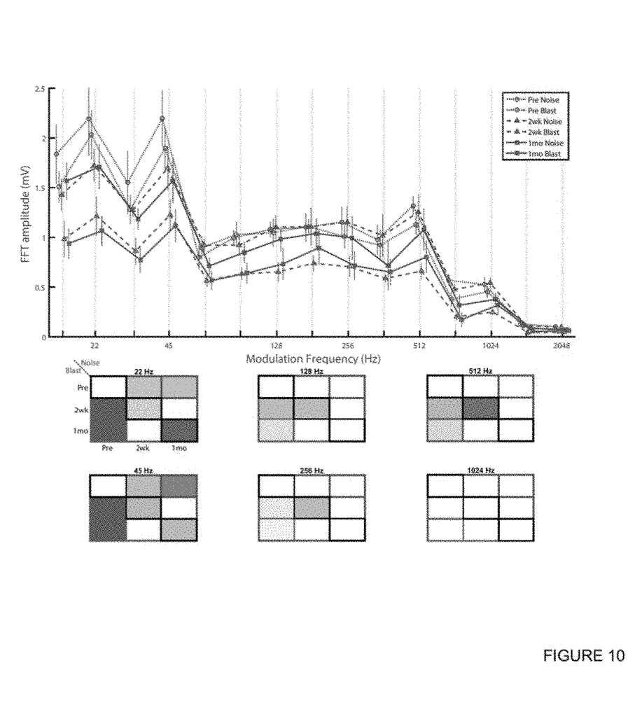

EFRs were recorded using an 8 kHz tone (SAM) carrier with sinusoidal amplitude modulation at 100% modulation depth. Fifteen amplitude modulation frequencies (AMFs) ranging from 16-2048 Hz in half-octave steps were tested in each recording session. All results are plotted on the left in FIG. 10, but for ease of statistical visualization, 6 representative AMFs were chosen to represent the range of AMFs tested (22, 45, 128, 256, 512, and 1024 Hz) on the right side of FIG. 10. These AMFs have been tested in previous EFR studies in aging animals (Parthasarathy and Bartlett 2011, 2012, Parthasarathy et al. 2010, 2014).

For EFRs of SAM at 80 dB SPL (FIG. 10), significant main effects of Group (F_{1,12}=15.59|p=0.002|ηₚ²=0.56), Time Point (F_{2,23=36.23}|p<0.0001|ηₚ²=0.76), and, unsurprisingly, Frequency (F_{14,168}=81.00|p<0.0001|ηₚ²=0.87) were observed. A significant Group*Time Point interaction effect was also observed (F_{2,23}=7.38|p=0.003|ηₚ²=0.39). At both 2 weeks and 1 month, the main effects indicate significantly lower EFR amplitudes in Blast compared to Noise animals. Lower modulation frequency recordings (≤64 Hz) at 2 weeks and 1 month in Blast animals revealed widespread significant EFR amplitude reductions (worse) when compared to pre-exposure recordings. Noise animals demonstrated trends of slight decrease (worse) for lower modulation frequency EFR amplitudes, but only a few were significant (ex: 45 Hz pre vs. 1 month). Blast EFR amplitudes were reduced (worse) when compared Noise EFRs at lower modulation frequencies (significant at 22 and 32 Hz; trend at 16, 45, and 64 Hz) for both 2 week and 1 month post-injury time points. Middle modulation frequency recordings (90-512 Hz) demonstrated trends of decrease (worse) for Blast animals, but not for Noise animals, when 2 weeks and 1 month were compared to pre-injury recordings. Some of the observed decreases were statistically significant (ex: 512 Hz). Noise animals did not demonstrate trends or significant changes at either 2 weeks or 1 month post-injury when compared to pre-injury recordings. Neither group demonstrated trends or significant differences for higher modulation frequencies (≥724 Hz).

The auditory system is dually susceptible to blast via a combined injury to both the PAS and CAS. The presented results detail our investigation of PAS and CAS function after exposure to a single blast (shock wave+noise) or noise-only acoustic trauma event. We hypothesized the dual insult intrinsic to the nature of blast exposure would cause a greater degree of hearing impairment in Blast animals than in Noise animals that would be detectable and differentiable with non-invasive auditory tests. This phenomenon was observable in our results, which widely demonstrated Blast animals having more severe, persistent auditory deficits in post-injury PAS and CAS assessments compared to Noise animals. The findings from the present study extend previous findings and are largely consistent with them. Previous animal studies have found hearing threshold increases (worse), impairments in gap detection, and compromised pre-pulse inhibition of startle following blast exposure, primarily for higher-frequency sounds (Luo et al 2014a, Mahmood et al. 2014). These changes were associated with OHC loss (Cho et al. 2013b, Ewert et al. 2012) and loss of hair cell/spiral ganglion synapses for IHCs and OHCs (Cho et al. 2013b). Those observations are consistent with the reduced (worse) DPOAE amplitudes for higher frequencies (FIG. 3) and lasting reduction (worse) of wave I amplitudes (FIGS. 6-7). At the single unit level, both the dorsal cochlear nuclei (DCN) and the IC have been reported to undergo post-blast increases in spontaneous activity for units sensitive to higher frequencies (Luo et al. 2014a,b), which could desynchronize responses and compromise wave IV and V of the ABR response, similar to our findings (FIGS. 6-8).

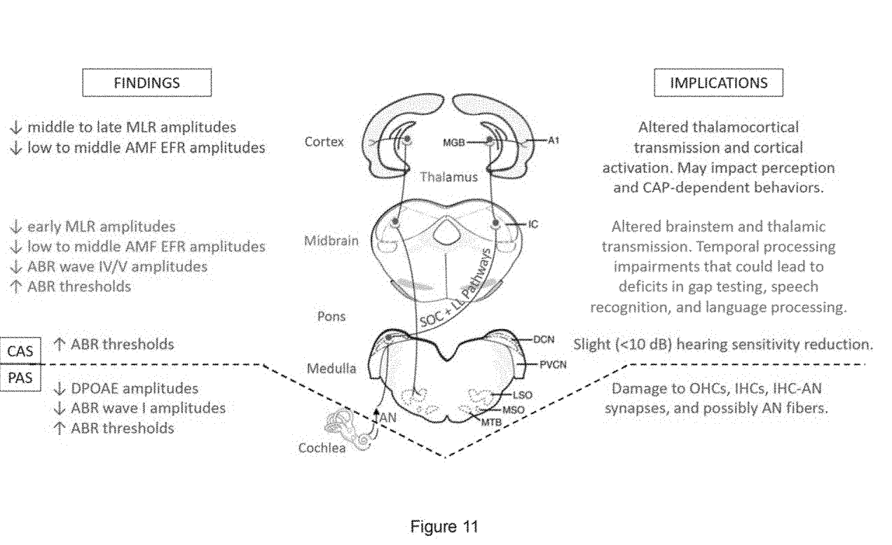

Notably, the impairments we observed were elicited by a relatively mild, single blast exposure, compared to more intense exposures in previous studies (Cho et al. 2013b, Du et al. 2013, Luo et al. 2014a,b, Mahmood et al. 2014). Further, most of the acoustic stimuli utilized in this and prior studies, specifically clicks and pure tones in the absence of background noise, are relatively simple in terms of CAP complexity needed for appropriate signal decomposition and response. It is possible and likely that more complex stimuli such as amplitude-modulated sounds and speech sounds, particularly with the addition of background noise, would reveal greater deficits. EFR results from this investigation and prior studies in aging support this notion (Anderson et al. 2012, 2013). Our results capturing significant post-blast impairments in CAP of temporally modulated sounds (FIG. 10) is an important step forward in this regard. The results of this investigation offer broad implications, summarized in FIG. 11, toward the scope of blast-induced auditory processing impairments which may impact battlefield performance, quality of life and societal reintegration, and future laboratory investigations of blast injury.

### Mild Blast Effects on the Peripheral Auditory System

DPOAE Amplitudes and OHC Integrity

The DPOAEs demonstrate differential injury effects on OHC performance dependent both on frequency (and thus basilar membrane locus) and injury type (Blast vs. Noise), as shown in FIGS. 2 and 3. Among the tested stimuli, noise-only injury appeared to cause selective damage to OHCs at the 8 kHz region, while blast exposure incited injury to OHCs at both 8 and 12 kHz. This suggests shock wave exposure indeed causes additional detriment to the PAS compared to conventional noise-induced acute acoustic trauma, even when the tympanic membrane remains structurally intact as in this model. A reduction in DPOAE amplitude indicates the possibility of either OHC death (complete loss of OHC) or OHC dysfunction (OHCs are present but have lost their function; Chen et al. 2009, Kemp 1978). DPOAE results from a prior study reported that the overall cochlear anatomy was normal but complete OHC loss was discovered in the cochlear base (Cho et al. 2013b), which corresponds to higher-frequency regions. This finding is similar to other reports describing blast-induced hearing loss in rats (Ewert et al. 2012) as well as to our studies in which DPOAEs were affected at 8 and 12 kHz, but not at 2 or 4 kHz (FIGS. 2, 3). We thus infer that blast-induced OHC dysfunction and/or death existed primarily at higher-frequency regions in Blast animals, consistent with previous reports (Cho et al. 2013b, Ewert et al. 2012). This may be a common feature of shock wave exposure independent of differing blast conditions between investigatory teams. For stimuli at or above 8 kHz, Blast and Noise pathological responses appear to diverge between 2 weeks and 1 month post-injury. With respect to pre-injury I/O functions, Noise animals became more sensitive (better) at 1 month while Blast animals were persistently impaired, suggesting underlying differences in pathophysiology and capacity for functional recovery between single event blast and noise-only acoustic trauma (FIG. 3). The findings of Luo et al. (2014a,b) agree with the notion that the pathological basis of blast- and noise-induced auditory processing deficits are fundamentally different.

Auditory Nerve Evoked Potential Thresholds and Response Amplitudes

Damage to OHC integrity naturally suggests downstream effects on ABR thresholds and wave amplitudes may be present. ABRs contain waveform features that correspond to specific generators within the auditory neuraxis. Evoked potential responses from the distal, extracranial fibers of the AN are represented in ABR wave I, where significant amplitude reductions were observed at 80 dB pSPL and 30 dB SL in Blast animals at all post-injury time points (FIGS. 4-5). Additionally, our results indeed indicated significant, but small (<10 dB), persistent threshold elevations (worse) in response to 0.1 msec broadband click stimuli (with similar trends for higher-frequency tone stimuli) at both 2 weeks and 1 month post-injury for Blast, but not Noise animals (FIG. 4). Threshold shifts <10 dB are less than many animal and human studies of aging or non-blast acoustic trauma (Henderson et al. 1994, Parthasarathy et al. 2014, Van Campen et al. 2002), and could be asymptomatic/subclinical. This observation is consistent with recent clinical reports of minimal changes in hearing sensitivity after blast in humans, despite the presence of behavioral alterations (Bressler et al. 2016, Gallun et al. 2012, Saunders et al. 2015).

Taken in context of DPOAE findings, the high-frequency threshold shifts in the audiograms suggest noise-only acoustic trauma principally impacts OHCs. Conversely, blast exposure demonstrates capacity to disrupt OHC and IHC integrity, synaptic coupling of IHCs to AN fibers, and perhaps even an overall reduction in AN fibers (Cho et al. 2013b). In higher-frequency tone data, a trend of diverging responses between groups was observed similar to the DPOAEs: the Blast group demonstrated threshold increases (worse), while decreases were observed in the Noise group (better). This could indicate a larger role for peripheral sensitization in the pathophysiological response to noise-only as compared to blast-induced acoustic trauma, again suggesting OHC and OHC+IHC damage in the Noise and Blast groups, respectively. Alternatively, as previously suggested by Ewert et al. (2012), it is possible that the heightened severity of blast trauma overwhelms the repair and remodeling capacity of the PAS, precluding development of peripheral compensatory recovery mechanisms. The persistence of Blast animals' significant post-injury 80 dB pSPL click wave I amplitude reductions with SL matching (30 dB SL) supports our hypothesis regarding the likely blast-induced functional disruption of synaptic connections between IHCs and AN fibers, for which some histologic evidence has been previously reported (Cho et al. 2013b), in addition to possible reduction of AN fibers. Conversely, SL-matched ABRs for the Noise group did not demonstrate significant differences from pre-injury levels, supporting the assertion that single event noise-only peripheral auditory deficits are primarily related to compromised integrity and function of OHCs, but not IHCs.

### Mild Blast Effects on the Central Auditory System

Click and Tone Processing Impairments

Longer latency waveforms in the click or brief pure tone evoked potential waveform correspond to the midbrain and brainstem auditory regions. The primary generators of ABR waves IV and V in rats are a combination of afferent inputs to the inferior colliculus (IC) including the superior olivary complex (SOC), lateral lemniscus (LL) and associated projections in the auditory midbrain (Blatchley et al. 1987, Chen and Chen 1991). Significant post-injury click wave IV, and V amplitude reductions (worse) were present in both Blast and Noise groups at 80 dB pSPL, but only in the Blast group at 30 dB SL (FIGS. 6-7). In contrast, 8 kHz tones at 80 dB pSPL did not reveal significant post-injury differences in wave IV or V amplitudes for either group (FIG. 8). The consistency of post-blast click threshold elevations (worse) and wave amplitude reductions (worse) could be a reflection of a click stimulus' broad coverage across a wide range of frequencies, particularly its inclusion of higher-frequency regions. The applied click stimuli induce broadband cochlear excitation encompassing all tested tone frequencies in addition to higher frequencies beyond 32 kHz. The click stimuli's broad inclusion of numerous higher-frequency regions, where more PAS damage was observed in this study (FIGS. 3, 5) and prior reports (Cho et al. 2013b, Ewert et al. 2012), is likely an asset for diagnostic purposes. The observed higher-frequency (16 and 32 kHz) tone threshold increases echo this notion in that, while somewhat variable, they were consistently elevated (worse) at all post-injury time points (FIG. 5). In contrast, lower-frequency (2-12 kHz) tone ABRs captured mixed, inconsistent, or a lack of post-injury threshold alterations (FIG. 5).

Based on the threshold and wave amplitude results, we suggest broadband click and higher-frequency (≥12 kHz) tone stimuli may be the most reliable simple stimuli for capturing post-blast ABR threshold shifts and wave amplitude alterations, particularly for wave IV and V generators. Clicks may prove especially useful given: 1) their inclusion of higher-frequency regions where more obvious changes were also observed in the tone results, and 2) while speculative, evidence supports the notion that blast-induced CAS injuries are likely multi-focal rather than diffuse (Budde et al. 2013, Cernak et al. 2001, Cho et al. 2013a, Garman et al. 2011, Readnower et al. 2010, Song et al. 2015, Walls et al. 2015). As such, after a single injury exposure, regions corresponding to some frequencies could be injured enough to elicit measureable ABR deficits, while other nearby areas retain relatively normal function. This type of multi-focal impairment likely differs on a subject-to-subject basis (even in the laboratory, albeit to a lesser degree than in the clinic) due to variability between blast exposures. The broad frequency coverage of clicks could help capture such heterogeneous, multi-focal injuries. In the same vein, narrowing a test's frequency coverage to a specific frequency (ie: using a pure tone stimulus), may be less likely to capture deficits in an individual subject.

The lack of major alterations in waves IV and V of the 8 kHz 80 dB pSPL tone ABR were surprising (FIG. 8), given the significant reductions in DPOAE I/O function amplitude (FIG. 3) and wave I amplitude (FIG. 8) at 8 kHz. Neuroplastic remodeling within CAS ABR generator regions is a reasonable hypothesis that could help explain this phenomenon. Indeed, neuroplastic changes in brainstem auditory nuclei have been widely reported in the hours, days, and weeks following acoustic trauma (Mulders et al. 2011, Mulders and Robertson 2009, 2011, 2013, Manzoor et al. 2013, Robertson et al. 2013). In the 2 weeks following injury, brainstem auditory evoked potential generators could remodel to preserve core CAP functions near the best hearing frequencies and compensate for compromised PAS integrity. Alternatively, it is possible that relatively simple stimuli such as pure tones near the rats' best hearing frequency were simply not sufficient to reveal substantial wave IV and V deficits, but wave I measurements were sensitive enough to detect changes. Of note, wave I measurements are also being pursued as diagnostic tools for ‘hidden hearing loss’ in humans, due to their high sensitivity and potential for objective evaluation (Plack et al. 2016). It is also possible that a larger sample size was needed to observe effects for tone measurements in waves IV and V, due to higher variability in more central generator responses.

Cho et al. (2013b) reported increased ABR thresholds across all tested frequencies immediately following injury and incomplete recovery of ABR thresholds in animals injured with blast pressures (123 and 181 kPa) similar to our study. They also reported least recovery of function at the highest and lowest frequencies. These findings are partially consistent with our ABR results, which captured incomplete recovery of ABR thresholds at higher frequencies (≥16 kHz) at 1-month post-blast but no threshold shift at lower frequencies (≤8 kHz) in Blast animals. Prior blast studies have reported ABR thresholds, but not wave amplitudes or latencies. This study is the first that reports blast effects on amplitudes of ABR wave I, IV and V elicited by clicks and brief tones (latencies were unaffected). As such, we are the first to directly demonstrate electrophysiological evidence of CAP dysfunction after a single blast exposure, which were significantly worse than deficits observed following a single noise-only acoustic trauma. Overall, the ABR wave deficits suggest altered signal processing between the AN and inputs to the IC.

Moving more centrally along the auditory neuraxis, MLR click and tone assessments provided a window into noise- and blast-induced alterations in thalamic, thalamocortical, and cortical processing. The slower presentation rate and longer analysis window for the MLR permit analysis of later responses and more central regions of the auditory hierarchy. While minimal changes in the MLR were observed for noise exposure alone, blast exposure incited lasting waveform amplitude changes in the latency windows corresponding to thalamocortical and cortical generators (FIG. 9), based on lesion studies in rats and other mammals (Barth and Di, 1991, Brett et al. 1996, Di and Barth 1992, Kraus et al. 1992, Kraus and McGee 1992, Phillips et al. 2011, Suta et al. 2011). While Mao et al. (2012) previously demonstrated region-specific blast-related damage using diffusion tensor imaging (DTI) in the IC and auditory thalamus of rats, but not the auditory cortex, cortex sparing can likely be attributed to non-equivalent injury mechanics from differing exposure conditions. Another DTI investigation found predominantly cortical changes in DTI results after blast exposure, including the auditory cortex (Budde et al. 2013). It is unclear from our work alone whether damage to auditory thalamic and cortical function results directly from the physical insult of blast, delayed secondary biological processes or a combination of both, but our physiological data are consistent with these anatomical data to strongly suggest that both subcortical and cortical auditory structures are susceptible to blast and result in functional deficits. As has been demonstrated in ototoxicity, noise-induced hearing loss, and aging studies, these deficits can propagate more centrally via maladaptive plasticity to result in deficits in higher auditory regions (in this case the cortex), even when those regions were not physically damaged (Mulders et al. 2011, Mulders and Robertson 2011, Salvi et al. 2000). In addition to PAS and brainstem blunted hearing sensitivity and general dysfunction, thalamus-cortex and cortex-cortex signaling alterations after blast (whether direct or secondary) are important to recognize as possible etiologic contributors toward eventual changes in perception and behavior, as have been observed by post-blast impairments in pre-pulse inhibition acoustic startle tests (Elder et al. 2012, Luo et al. 2014, Mao et al. 2012). In Veterans exposed to blast, cortical impairments have been directly implicated as a source of lasting impairment in an auditory attentional tasks, as peripheral sensory deficits could not explain the deficits (Bressler et al. 2016). Collectively, the DPOAE, ABR, and MLR data strongly suggest blast exposure compromises normal electrophysiological function, even to relatively simple stimuli such as clicks and pure tones, at all levels of the auditory neuraxis ranging from the organ of Corti to the auditory cortex. Given the simplicity of the tested stimuli it is possible and likely, as our EFR results demonstrate, that more challenging processing tasks could elicit more dramatic CAP deficits.

### Temporal Modulation Processing Impairments

ABRs, evoked by click or brief tone stimuli, provide phasic (onset) information regarding hearing thresholds, neural synchronization and activation, and neural signal transmission along the auditory pathway (Chen and Chen 1991, Hall 2007, Rowe III 1981, Thompson et al. 2001). However, human speech and other complex sounds are longer in duration (ten to hundreds milliseconds) and are rapidly modulated in amplitude as well as frequency over time (Rosen, 1992). To gain information about sustained responses and temporal modulation processing of the central auditory pathway, EFRs are useful because they reflect the faithfulness of the auditory subcortices in encoding and phase-locking to the modulation envelope of complex sounds (Aiken and Picton 2008a,b, Clinard et al. 2010, Cunningham et al. 2001, Parthasarathy and Bartlett 2011, Picton 2003). In fact, EFRs are the summed synchronized responses (spiking activity and synaptic potentials) of populations of neurons in the auditory brainstem and midbrain (Chandrasekaran and Kraus 2010, Herdman et al. 2002, Kuwada et al. 2002, Parthasarathy and Bartlett 2012, Picton et al. 2003). From more central (cortex) to more peripheral (auditory nerve) anatomical locations, each contributor to the EFR signal principally encodes increasingly higher AMFs (Gaese and Ostwald 1995, Paolini et al. 2001, Rees and Moller 1983, Rees and Moller 1987). This phenomenon has been observed across numerous species and has been well-summarized by Joris et al. (2003).

Blast group EFR deficits were observed primarily in the lower range of AMFs tested (≤64 Hz) without major differences at higher AMFs (corresponding to lower generator regions), suggesting the IC may be the primary source of blast-induced temporal processing deficits in this study (FIG. 10). Additionally, ABR waves IV and V (inputs to the IC) for the pure, unmodulated carrier tone (8 kHz, 80 dB SPL) did not capture significant differences from pre-injury recordings in either amplitude (FIG. 8) or latency (data not shown). These results support the notion that alterations within the IC itself, not preceding conduction or envelope coding failures, were the principal etiologic contributors to the observed reduction in EFR amplitudes. However, potential contributions of thalamocortical damage cannot be ruled out (FIG. 9). Deficits suggesting IC dysfunction are consistent with our prior assertion from the MLR data that subcortical auditory structures may be particularly vulnerable to blast, as was reported previously via DTI abnormalities in the IC and MGB (Mao et al. 2012). Furthermore, both the DCN and IC have been reported to demonstrate spontaneous hyperactivity after blast exposure in rats; however, only the IC sustained long-term hyperactivity (Luo et al. 2014 a,b).

CAP deficits in the auditory midbrain were more readily observable in 8 kHz SAM EFRs (FIG. 10, AMFs≤64 Hz) than pure tone 8 kHz ABRs (FIG. 8, Waves IV and V), reinforcing our assertion that complex stimuli may be more sensitive to CAP deficits after mild blast exposure than clicks and pure tones. More broadly, the lower AMF deficits in SAM EFRs but not pure tone ABRs imply that CAS neuroplasticity after blast exposure can compensate to some degree, perhaps enough to pass as ‘within normal limits’ on simple click and pure tone threshold or ABR tests. However, more complex stimuli may have higher sensitivity for detection of ‘silent’ subclinical (asymptomatic/non-perceived) CAS dysfunction. Indeed, perceptual and behavioral deficits have been reported in blast-exposed Veterans in absence of major threshold or ABR abnormalities (Bressler et al. 2016, Gallun et al. 2012). In rats, deficits on post-blast gap testing, a behavioral test dependent on temporal processing, have also been widely reported despite inconsistent mechanism-related observations regarding the presence of tinnitus, DPOAE threshold changes, or other potential etiologic contributors (Luo et al. 2014a,b, Mahmood et al. 2014, Mao et al. 2012). Our observations of post-blast EFR deficits in the IC and altered thalamocortical MLRs could help explain previous reports of impaired gap test performance in blast-exposed animals despite inconsistent PAS observations (Luo et al. 2014a,b, Mahmood et al. 2014, Mao et al. 2012). Impairments in midbrain temporal processing and thalamocortical transmission could certainly impact a subject's performance in a gap testing paradigm by disrupting the ability to perceive and respond appropriately to the stimuli. Such deficits could result from progressive centralization of compensatory remodeling in the auditory system after acoustic trauma (Mulders et al. 2011, Mulders and Robertson 2011), direct blast-induced damage to CAS structures (Budde et al. 2013, Cernak et al. 2001, Cho et al. 2013a, Garman et al. 2011, Readnower et al. 2010, Walls et al. 2015), or (most likely) a combination of both pathophysiological processes. Of note, a clinical investigation of blast-exposed Veterans did not find consistent evidence of subcortical EFR deficits (although small amplitude reductions were present), and cortical networks were implicated as principally responsible for auditory attentional task impairments (Bressler et al. 2016). While somewhat in contrast to our EFR findings, this likely owes to the high variability in number and type of blast exposures in uncontrolled battlefield environments as well as the wide range of auditory brainstem responses in human subjects, underscoring the importance of laboratory investigations to supplement clinical findings.

### Implications for Blast-exposed Individuals and Future Laboratory Studies

Study of blast injury to the brain is a young field, having gained significant popularity and funding opportunities stemming from military operations in Middle Eastern conflicts over the last two decades. Despite significant effort, mild injuries remain elusive from both a diagnostic and treatment perspective, in large part due to lack of obvious symptoms and unremarkable imaging findings (Prevention NCfI, 2013). As demonstrated in this study, the exquisite sensitivity of the auditory system to even mild bTBI highlights it as a promising avenue for development of new diagnostic tests. Our results suggest numerous rapid, non-invasive AEP assessments (ABRs, MLRs, and EFRs) appear sensitive to CAP changes after blast exposure. As such, AEPs may present a promising avenue for future investigation in both animal and human studies.

Beyond diagnosis, the intertwined nature of auditory processing with critical activities for optimal battlefield performance and for successful civilian re-entry opens new avenues for research that may benefit blast-injured individuals. Heightened understanding of how blast-induced CAP alterations affect learning, memory, communication, and emotional responses could play an important role in shaping new therapies ranging from devices to drugs to psychotherapy, which have been reported to have reduced efficacy in the Veteran population (Institute of Medicine 2014). Hearing and auditory processing are an integral part of daily life, both on the battlefield and upon societal reintegration, which underscores the importance of protecting, rather than restoring, both PAS and CAS function of current and future service personnel. To this end, the military has begun rollout of the Tactical Communication and Protective System (TCAPS), a damped ‘smart’ earplug designed to protect the PAS from impulse noise-induced hearing impairment and enhance battlefield communication (Casali et al 2009, Clasing and Casali 2014). Further measures will be needed to confer protection against direct TBI-induced (with or without blast) CAS damage in the brain and brainstem, as highlighted in this study.

There are many functional networks in the brain that, to complete certain tasks or respond to stimuli appropriately, are intertwined with auditory processing. Many of these networks are of interest in bTBI laboratory investigations. Post-blast learning, memory, fear, and emotional alterations have emerged as high-interest topics due to the elevated reporting frequency of such deficits among the Veteran population (Carlson et al. 2010, Elder et al. 2012, Goldstein et al. 2012, Maguen et al. 2012, Tanielian et al. 2008). In the laboratory setting, rodent behavior to assess such changes is often studied in one of many paradigms in which a set of stimuli, often acoustic, correspond to subsequent events that rats can be trained to perform or respond to. This phenomenon is known as cued operant conditioning and is commonly used in working memory tasks or fear conditioning paradigms (Goosens and Maren 2001, Heldt et al. 2014, Sakurai 1994). Based on the findings of this study and with the understanding that auditory processing is often a critical component for successful completion of these paradigms, it is prudent to avoid using stimuli for which auditory processing impairments could confound interpretation of paradigm performance. If non-auditory brain regions are the primary investigatory target(s), non-acoustic alternatives such as light, odor, or tactile stimuli may be more suitable operant task discriminants in the context of mild bTBI.

Taken together, the data from this study strongly suggest blast exposure compromises normal auditory processing functions at all levels of the auditory neuraxis ranging from the organ of Corti to the auditory cortex (FIG. 11) to a greater degree than non-blast acoustic trauma. We observed particular difficulty in the processing of temporally modulated sounds, suggesting blast-induced CAP deficits may become more dramatic with increasingly complex processing tasks such as sound localization, speech and non-speech sound recognition in noise, or language processing. With this study we reinforce Luo et al's assertion (2014a,b) that acute blast exposure and noise exposure incite fundamentally different pathologies which may require distinct interventions to prevent or treat resultant auditory processing impairments. The contributions of primary (mechanical) and secondary (biological/biochemical) damage should be elucidated. While the prior work of us and others in blast-induced TBI would suggest a multi-focal primary+secondary injury to numerous brain and brainstem CAS regions (Budde et al. 2013, Cernak et al. 2001, Cho et al. 2013a, Garman et al. 2011, Readnower et al. 2010, Song et al. 2015, Walls et al. 2015), further research in this model using biochemical, immunohistochemical, and in-vivo imaging techniques is warranted to directly expound upon the biophysical basis of the observed deficits. Specifically, efforts should be targeted toward discovering the initial anatomical contributor(s) to post-blast auditory processing deficits and documenting pathological progression throughout the auditory neuraxis. These efforts may help disentangle the mechanisms and time course of the differential contributions of 1) direct blast-induced damage to the CAS, and 2) the progressive centralization of secondary maladaptive plasticity (stemming from PAS dysfunction) that is known to occur after AAT (Mulders and Robertson 2011). If the root mechanisms linking the peripheral and central physical insults to electrophysiological deficits and subsequent behavioral changes are better understood, there exists great opportunity for well-directed development of protective and therapeutic innovations.

### Significant Elevation of Acrolein-Adduct in Auditory Cortex Examined 2, and 7 Days After Blast Trauma

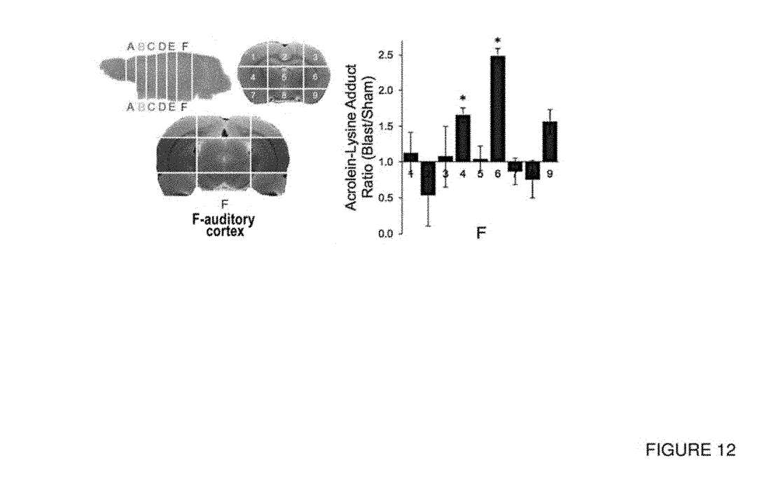

We have shown previously that acrolein causes significant oxidative damage in isolated mitochondria, PC12 neural progenitor cells, and spinal cord segments by disrupting myelin and membrane integrity and inhibiting key mitochondrial enzymes, all of which lead to oxidative stress and exacerbate neuroinflammation. We have also shown that acrolein is elevated in mbTBI, particularly in auditory thalamus, and auditory cortex, key components of central auditory neuraxis (FIG. 12).

### Post Blast Functional Recovery Window Period

FIG. 13 indicates pitch contours generated by iterated rippled noise (IRN) waveforms. Speech like temporal fine structure is significantly diminished immediately after blast impact. The top panel of FIG. 13 shows waveform and spectrogram of the speech-like sound are used to probe complex sound representations. In the bottom panel of FIG. 13, example auditory evoked potential waveforms 7 days post-noise (blue) or blast (red) exposure are shown for an increase in the iterations or pitch salience going from top to bottom. In all cases, the blue waveform is larger and much closer to the stimulus waveform than the blast exposed responses. This indicates that post-blast day 7-10 seems to be the period of rapid functional recovery from initial damage, as shown in ABR threshold and amplitudes.

1. A standalone device for evaluating brain injury, comprising:
an apparatus to provide and measure a controlled sound stimuli, and
at least one single or multichannel auditory evoked potential recordings of synchronized neural activities selected from the group consisting of distortion product otoacoustic emissions (DPOAE), auditory brainstem responses (ABRs), envelope following responses (EFRs), and middle-latency responses (MLRs).

2. The standalone device according to claim 1, wherein the controlled sound stimuli is selected from noises comprising simple clicks and pure tones, and temporally modulated sounds.

3. A method of evaluating brain injury in a patient, comprising:
a. using a standalone device to measure the baseline single or multichannel auditory evoked potential recording of synchronized neural activities selected from the group consisting of distortion product otoacoustic emissions (DPOAE), auditory brainstem responses (ABRs), envelope following responses (EFRs), and middle-latency responses (MLRs);
b. monitoring the patient until a sound event occurs;
c. using the standalone device to measure the post event auditory evoked potential recording of synchronized neural activities identified in step a at an extended time point;
d. determining the auditory evoked potential recording differences for DPOAE, ABRs, EFRs and MLRs between the baseline and post event time point; and
e. correlating DPOAE, ABRs, EFRs and MLRs data associated brain region to determine the extent of brain injury.

4. The method according to claim 3, wherein the extent of brain injury includes brain stem, thalamus and cortex.

5. The method according to claim 3, further comprising examining acrolein-adduct in auditory cortex at about day 2 and about day 7 post event, wherein increased acrolein in auditory cortex and auditory thalamus indicates brain injury.

6. A method of non-invasively evaluating a patient's post event auditory recovery, comprising:
providing a sound speech to the patient at various time point after the event;
obtaining a waveform or a spectrogram of the patient;
ascertaining a clear iteration and/or pitch salience of each sound speech provoked; and
recording the time point when a peak iteration and/or a pitch salience is identified.

7. The method according to claim 6, wherein the time point is about 7 days post a blast.
